(19) United States
(12) Patent Application Publication
(10) Pub. No.: US 2009/0258966 A1
(43) Pub. Date: Oct. 15, 2009

(54) Dental Composition

(75) Inventors: Satoshi Hirayama, Tokyo (JP); Takuji Ikemi, Tokyo (JP); Yasufumi Tsuchiya, Chiba (JP); Tatsuya Ori, Shiga (JP); Takashi Yamamoto, Shiga (JP)

Correspondence Address: THE WEBB LAW FIRM, P.C., 700 KOPPERS BUILDING, 436 SEVENTH AVENUE, PITTSBURGH, PA, 15219, US

(73) Assignees: NIHON UNIVERSITY, Tokyo (JP); SUN MEDICAL CO., LTD., Moriyama-shi (JP)

(21) Appl. No.: 12/089,334

(22) PCT Filed: Oct. 4, 2006
(86) PCT No.: PCT/JP2006/319896

§ 371 Date: Apr. 4, 2008

(30) Foreign Application Priority Data
Oct. 4, 2005 (JP) 2005-291378
Oct. 4, 2005 (JP) 2005-291379

(51) Int. Cl. A61K 6/00; C08K 3/32

(52) U.S. Cl. 523/118; 523/105

(57) ABSTRACT

An object of the present invention is to provide a dental composition having excellent adhesive effect and simultaneously not causing microleakage.

A dental composition of a first embodiment of the present invention includes (A) an acidic-group containing polymerizable monomer, (B) a polymerizable monomer, (C) a filler comprising poly(meth)acrylate particles, (D) a calcium-containing material including tetracalcium phosphate (TTCP) and dicalcium phosphate (DCP), and (E) a polymerization initiator. The component (A) is contained at 16-70 parts by weight based on a total of 100 parts by weight of (A)+(B). The component (B) is contained at 84-30 parts by weight based on a total of 100 parts by weight of (A)+(B). The component (C) is contained at 0.2-297 parts by weight relative to a total of 100 parts by weight of (A)+(B). The component (D) is contained at 0.2-297 parts by weight relative to a total of 100 parts by weight of (A)+(B).

### FIELD OF THE INVENTION

The present invention relates to a dental composition. More specifically, the present invention relates to a dental composition able to be used in a dental cement, a dental bonding material, etc.

### BACKGROUND OF THE INVENTION

Dental caries is a disease in which the teeth undergo decalcification by acids produced by dental caries-causing bacteria present in the oral cavity, and treatment for this disease is performed by grinding out the site of infection and filling/repairing with a dental material. These treatments are carried out using adhesive materials such as adhesive resin cements, bonding materials or glass ionomer cements.

For adhesion to teeth, importance is placed not only on initial adhesive strength, but also on durability in the oral cavity environment. For example, in the oral cavity environment, if microleakage occurs due to a gap appearing at the dentin-adhesive material boundary caused by a reduction in adhesive effect, irritant substances and germs often infiltrate via this gap, and there is a high probability that dental caries can recur at the repaired part, meaning that the treatment is not successful.

On the other hand, in addition to the conventional dental adhesive materials mentioned above, non-patent document 1, etc. proposes the use of an adhesive material which has calcium phosphates (tetracalcium phosphate and dicalcium phosphate) as primary components, and which is converted into a hydroxyapatite similar to living hard tissue. However, there are concerns that the scope of application is limited from the view point of initial adhesive strength, etc., particularly in the treatment of dental caries.

With an object of improving biocompatibility and mechanical strength, various dental adhesive compositions and bioadhesive compositions containing calcium phosphates have been proposed. However, if dental adhesives are used together with these inorganic compounds, there are concerns that the adhesive performance will be greatly reduced.

Patent document 1, etc. proposes a composition comprising a calcium phosphate powder, a polymethacrylate powder and a methacrylate monomer. Although this composition can give a cured product having excellent physical properties, etc., the composition cannot adhere to dentin or hard tissue with high adhesive strength and may cause microleakage.

Therefore, a dental composition having excellent adhesive effect and simultaneously not causing microleakage is desirable.

[Non-patent document 1] Journal of Hard Tissue Biology; 4(1): 1-7

[Patent document 1] Japanese Unexamined Patent Application Publication No. 2001-231848

### DISCLOSURE OF THE INVENTION

### Problems to be Solved by the Invention

An object of the present invention is to provide a dental composition having excellent adhesive effect and simultaneously not causing microleakage.

### SUMMARY OF THE INVENTION

A dental composition according to a first embodiment of the present invention comprises:

(A) a compound having at least 1 acidic group and polymerizable group in the molecule,

(B) a compound having 1 polymerizable group, but not having an acidic group, in the molecule,

(C) a filler comprising poly(meth)acrylate particles,

(D) a calcium-containing material including tetracalcium phosphate (TTCP) and dicalcium phosphate (DCP), and

(E) a polymerization initiator,

the component (A) being contained at 16-70 parts by weight based on a total of 100 parts by weight of the components (A) and (B), the component (B) being contained at 84-30 parts by weight based on a total of 100 parts by weight of the components (A) and (B), the component (C) being contained at 0.2-297 parts by weight relative to a total of 100 parts by weight of the components (A) and (B), the component (D) being contained at 0.2-297 parts by weight relative to a total of 100 parts by weight of the components (A) and (B).

A dental composition according to a second embodiment of the present invention comprises:

(A) a polymerizable monomer having at least 1 acidic group in the molecule,

(J) a polymerizable monomer having at least 1 hydroxyl group in the molecule,

(K) a compound represented by formula (1) and/or formula (2) below,

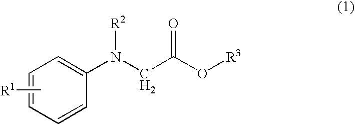

(wherein R¹ and R² represent each independently a hydrogen atom or an alkyl group optionally containing a functional group or a substituent group; and R³ represents a hydrogen atom or a metal atom);

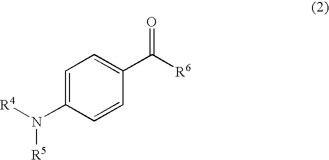

(wherein, R⁴ and R⁵ are each independently a hydrogen atom or an alkyl group; and R⁶ is a hydrogen atom, an alkyl group or alkoxyl group optionally containing a functional group or a substituent group), and

(D) a filler including tetracalcium phosphate (TTCP) and dicalcium phosphate (DCP),

the usage amount of the acidic group-containing polymerizable monomer (A) which constitutes the dental composition being 1-50 wt. %, the usage amount of the acidic group-containing polymerizable monomer (J) being 1-98.99 wt. %, the usage amount of the compound (K) represented by formula (1) and/or formula (2) being 0.01-30 wt. %, each relative to 100 wt. % of the total usage amount of the acidic group-containing polymerizable monomer (A), the hydroxyl group-containing polymerizable monomer (J) and the compound (K) represented by formula (1) and/or formula (2),

the usage amount of the calcium filter (D) including tetracalcium phosphate (TTCP) and dicalcium phosphate (DCP) being 15-95 parts by weight relative to 100 parts by weight of the total usage amount of the acidic group-containing polymerizable monomer (A), the hydroxyl group-containing polymerizable monomer (J) and the compound represented by formula (1) and/or formula (2).

### ADVANTAGES OF THE INVENTION

The dental compositions of the present invention have excellent adhesive effect in the treatment of dental caries and are also effective in preventing microleakage.

### DETAILED DESCRIPTION OF THE INVENTION

A detailed description of the dental compositions of the present invention will now be given. Hereafter, “parts” and “%” are given by weight unless otherwise designated.

The dental compositions of the invention may be polymerizable dental adhesive compositions containing calcium phosphate compounds. In more detail, the calcium phosphate compounds are used as fillers in the polymerizable dental adhesive compositions. The calcium phosphate compounds are converted into a hydroxyapatite similar to living hard tissue and form a biological part, whereby the compositions provide strong adhesion to dentin and hard tissue, which are adherends. Furthermore, the compositions also exhibit good adhesive strength to dental prostheses such as dental composite resins, dental hard resins, ceramics, and dental metals including gold-silver-palladium alloys. The compositions can thus prevent microleakage.

The dental compositions of the present invention are roughly classified into a first embodiment and a second embodiment.

A description of the dental composition of the first embodiment will now be given.

The component (A) that is blended in the dental composition of the first embodiment of the present invention, is a polymerizable compound containing at least 1 acidic group and polymerizable group in the molecule. Radical polymerizable groups are preferably used as the polymerizable groups in the component (A), and examples include vinyl groups, vinyl cyanide groups, acryloyl groups, methacryloyl groups, acrylamide groups, methacrylamide groups, etc. Examples of the acidic groups include carboxyl groups, phosphate groups, thiophosphate groups, sulfo groups, sulfino groups, etc. In addition, the acidic groups include groups which substantially function as acidic groups by being easily decomposed under working conditions to form the acidic groups, with examples including carboxylic acid anhydride groups.

In the present invention, examples of the polymerizable compounds having carboxyl groups, which are specific examples of the components (A), include:

α-unsaturated carboxylic acids such as (meth)acrylic acid (hereafter, (meth)acrylic acid is used to denote acrylic acid and methacrylic acid.) and maleic acid;

vinyl aromatic compounds such as 4-vinyl benzoic acid;

carboxylic acid compounds in which a linear hydrocarbon group is between a (meth)acryloyloxy group and a carboxylic acid group, such as 1′-(meth)acryloyloxy-1,1-undecanedicarboxylic acid (hereafter, (meth)acryloyl is used to denote acryloyl and methacryloyl.);

(meth)acryloyloxyalkylnaphthalene(poly)carboxylic acids such as 6-(meth)acryloyloxyethylnaphthalene-1,2,6-tricarboxylic acid;

(meth)acryloxyalkyltrimellitic acids such as 4-(meth)acryloxymethyltrimellitic acid, 4-(meth)acryloxyethyltrimellitic acid and 4-(meth)acryloxybutyltrimellitic acid;

compounds as described above which further contain hydroxyl groups such as 4-[2-hydroxy-3-(meth)acryloyloxy]butyltrimellitic acid;

compounds having carboxybenzoyloxy groups such as 2,3-bis(3,4-dicarboxybenzoyloxy)propyl (meth)acrylate (hereafter, (meth)acrylate is used to denote acrylate and methacrylate.);

mono(meth)acryloyloxyamino acids or di(meth)acryloyloxyamino acids substituted at the N- and/or O-position, such as N,O-di(meth)acryloyloxytyrosine, O-(meth)acryloyloxytyrosine, N-(meth)acryloyloxytyrosine, N-(meth)acryloyloxyphenylalanine, O-(meth)acryloyloxyphenylalanine and N,O-di(meth)acryloyloxyphenylalanine;

(meth)acryloyl compounds of benzoic acids having functional substituent groups, such as N-(meth)acryloyl-4-aminobenzoic acid, N-(meth)acryloyl-5-aminobenzoic acid, 2-, 3- or 4-(meth)acryloyloxybenzoic acid and 4- or 5-(meth)acryloylaminosalicylic acid;

adduct reaction products of hydroxyalkyl (meth)acrylates and unsaturated polycarboxylic acid anhydrides, such as adduct reaction product of 2-hydroxyethyl (meth)acrylate and pyromellitic dianhydride, and adduct reaction product of 2-hydroxyethyl (meth)acrylate and maleic anhydride, 3,3′,4,4′-benzophenonetetracarboxylic dianhydride or 3,3′,4,4′-biphenyltetracarboxylic dianhydride; and

compounds having polycarboxybenzoyloxy and (meth)acryloyloxy groups, such as 2-(3,4-dicarboxybenzoyloxy)-1,3-di(meth)acryloyloxypropane, adduct of N-phenylglycine or N-tolylglycine and glycidyl (meth)acrylate, 4-[(2-hydroxy-3-(meth)acryloyloxypropyl)amino]phthalic acid, and 3- or 4-[N-methyl-N-(2-hydroxy-3-(meth)acryloyloxypropyl)amino]phthalic acid. Of these, 11-(meth)acryloyloxy-1,1-undecanedicarboxylic acid, 4-(meth)acryloxyethyltrimellitic acid, and anhydrides thereof are preferably used.

For example, phosphate ester groups having 1 or 2 hydroxyl groups can be preferably used as groups wherein at least 1 hydroxyl group is bonded to a phosphorus atom and as functional groups capable of being easily converted to such groups in water. Examples of the polymerizable monomers containing such groups include (meth)acryloyloxyalkyl acid phosphates such as 2-(meth)acryloyloxyethyl phosphate, 2- and/or 3-(meth)acryloyloxypropyl phosphate, 4-(meth)acryloyloxybutyl phosphate, 6-(meth)acryloyloxyhexyl phosphate, 8-(meth)acryloyloxyoctyl phosphate, 10-(meth)acryloyloxydecyl phosphate and 12-(meth)acryloyloxydodecyl phosphate;

acid phosphates having 2 or more (meth)acryloyloxyalkyl groups, such as bis[2-(meth)acryloyloxyethyl]phosphate and bis[2- or 3-(meth)acryloyloxypropyl]phosphate; and

acid phosphates containing a (meth)acryloyloxyalkyl group and an aromatic ring such as a phenylene group optionally via a hetero atom such as an oxygen atom, such as 2-(meth)acryloyloxyethylphenyl phosphate and 2-(meth)acryloyloxyethyl-p-methoxyphenyl phosphate. In these compounds, the phosphate groups may be replaced by thiophosphate groups. Of these, 2-(meth)acryloyloxyethyl phosphate is preferably used.

Examples of the polymerizable monomers having sulfo groups or functional groups that can be easily converted to sulfo groups in water include sulfoalkyl (meth)acrylates such as 2-sulfoethyl (meth)acrylate, 2- or 1-sulfo-1- or 2-propyl (meth)acrylate, and 1- or 3-sulfo-2-butyl (meth)acrylate;

compounds as described above wherein the alkyl groups have atom groups containing a hetero atom such as a halogen or oxygen, such as 3-bromo-2-sulfo-2-propyl (meth)acrylate and 3-methoxy-1-sulfo-2-propyl (meth)acrylate;

compounds as described above that have an acrylamide group instead of the acrylate group, such as 1,1-dimethyl-2-sulfoethyl (meth)acrylamide and 2-(meth)acrylamido-2-methylpropane sulfonic acid; and

vinyl aryl sulfonic acids such as 4-styrene sulfonic acid and 4-(prop-1-en-2-yl)benzene sulfonic acid. Of these, 4-styrene sulfonic acid is preferably used. These compounds may be used singly or as a combination of 2 or more thereof.

The usage amount of the component (A) in the dental composition of the first embodiment of the present invention is 16-70 parts by weight, preferably 18-50 parts by weight, and more preferably 20-40 parts by weight, based on a total of 100 parts by weight of this component (A) and component (B), which is mentioned later. By using this amount of the polymerizable monomer (A) which contains an acidic group and polymerizable group in the molecule, the dental composition of the first embodiment of the present invention shows good adhesion to hard tissue such as dentin, which is an adherend.

The component (B) contained in the dental composition of the first embodiment of the present invention is a compound containing 1 polymerizable group but not containing an acidic group in the molecule. Examples of the compounds which can be used as the component (B) include alkyl (meth)acrylates such as methyl (meth)acrylate, ethyl (meth)acrylate, n-propyl (meth)acrylate and n-butyl (meth)acrylate;

hydroxyalkyl (meth)acrylates such as 2-hydroxyethyl (meth)acrylate and 3-hydroxypropyl (meth)acrylate;

poly(alkylene glycol) alkyl ether (meth)acrylates (R¹—(—O—R²—)ₙ—O—COCH(R³)═CH₂ (R¹: alkyl group R²: alkylene group, R³: hydrogen or a methyl group) such as 2-(2-methoxyethoxy)ethyl (meth)acrylate and 2-(2-(2-(2-methoxyethoxy)ethoxy)ethoxy)ethyl (meth)acrylate;

acetoacetoxyethyl (meth)acrylate;

cycloalkyl (meth)acrylates such as cyclobutyl (meth)acrylate and cyclohexyl (meth)acrylate;

cyclic alkyl (meth)acrylates containing a hetero atom, such as (tetra hydrofuran-2-yl) (meth)acrylate;

(meth)acrylic acid fluoroalkyl esters such as perfluorooctyl (meth)acrylate and hexafluoroisopropyl (meth)acrylate; and

silane compounds containing (meth)acryloxyalkyl groups, such as 3-(trimethoxysilyl)propyl (meth)acrylate. These monomers may be used singly or as a combination of 2 or more thereof.

In order to obtain high adhesive strength to hard tissue such as dentin, it is preferable to use a low molecular weight monomer, for example, a low molecular weight monomer having a molecular weight of 300 or less, whereby the monomers can diffuse into the adhesive boundary with the dentin, etc. Examples of such low molecular weight monomers include methyl (meth)acrylate, ethyl (meth)acrylate, propyl (meth)acrylate, and ethylene glycol monomethyl ether (meth)acrylate. This type of low molecular weight monomer can be used as the polymerizable monomer component (B) itself.

In the present invention in particular, methacrylates that have relatively low irritation to the human body are preferably used as the component (B) that contains 1 polymerizable group but does not contain an acidic group in the molecule.

The component (B) is preferably capable of dissolving the component (A) that contains an acidic group, at ambient temperature.

The component (B) in the present invention is contained at 84-30 parts by weight, preferably 82-50 parts by weight, and particularly preferably 80-60 parts by weight, based on a total of 100 parts by weight of the polymerizable monomer component (A) containing an acidic group, and this component (B). The component (B) used in the present invention is a basic monomer essential to achieve the excellent properties of the dental composition of the first embodiment of the present invention. Using this component within the above-mentioned range establishes the fundamental characteristics related to the physical properties of the dental composition of the first embodiment of the present invention.

The total weight of the components (A) and (B) is preferably 0.14-99.1 parts by weight, more preferably 0.16-98.9 parts by weight, and still more preferably 0.17-98.4 parts by weight, based on 100 parts by weight of the composition of the present invention. If the total weight falls below the lower limit of the above-mentioned numerical range, handleability is dramatically reduced. If the total weight exceeds the upper limit of the above-mentioned numerical range, it is difficult to achieve desired adhesive performance.

The component (C) blended in the dental composition of the first embodiment of the present invention is a filler comprising poly((meth)acrylate) particles. Examples of the monomers which form the polymer particles include (meth)acrylate monomers such as alkyl (meth)acrylates such as methyl (meth)acrylate, ethyl (meth)acrylate, propyl (meth)acrylate and butyl (meth)acrylate, cycloalkyl (meth)acrylates such as cyclohexyl (meth)acrylate, and aromatic (meth)acrylates such as benzyl (meth)acrylate. The (meth)acrylate polymers in the present invention may be copolymerized with small quantities of crosslinkable monomers as required. For example, polyfunctional monomers such as ethylene glycol di(meth)acrylate, triethylene glycol di(meth)acrylate and butadienes can be used as the crosslinkable monomers.

The weight average molecular weight of the poly((meth)acrylate) particles preferably used as the filler (C) in the present invention is preferably 50,000-300,000.

The fillers (C) in the present invention may be used singly or as a combination of 2 or more thereof, or copolymers with the above-mentioned monomers may be used.

In the present invention, the average particle diameter of the particles of the filler (C) is preferably 0.001-30 μm, and particularly preferably 0.01-25 μm, in order to reduce the coating thickness and improve the repairing effect of the dental composition.

The filler (C) comprising the poly((meth)acrylate) particles in the dental composition of the first embodiment of the present invention can be used at 0.2-297 parts by weight, and preferably 0.3-248 parts by weight, based on a total of 100 parts by weight of the components (A) and (B) which form the dental composition of the first embodiment of the present invention.

The component (D) in the dental composition of the first embodiment of the present invention is a calcium-containing material including tetracalcium phosphate (Ca₄(PO₄)₂O) (hereafter designated as TTCP) and dicalcium phosphate (CaHPO₄) (hereafter designated as DCP). By using TTCP and DCP in combination, both TTCP and DCP are converted together with moisture in the oral cavity into a hydroxyapatite similar to dental hard tissue after the dental composition is applied, thereby providing stronger adhesion. For information, DCP is also known as dicalcium phosphate, calcium hydrogen phosphate, dicalcium hydrogen phosphate, secondary calcium phosphate and calcium monohydrogen phosphate, but all of these are the same, CaHPO₄. Moreover, these are preferably anhydrates, and the compositional weight ratios thereof are also calculated on this assumption.

The molar ratio of TTCP and DCP (TTCP/DCP) is normally 0.33-3.5, preferably 2.3-3, and particularly preferably 2.7. If this range is exceeded, the conversion into hydroxyapatite is insufficient and not only is the objective not achieved, but also the molecules which are not converted into hydroxyapatite react with the acidic groups in the polymerizable monomer component (A), resulting in dramatically reduced adhesion to the dentin, and further causing the mechanical properties of a cured product of the composition to deteriorate.

In the present invention, the form of the calcium-containing material (D) is not particularly restricted. For example, a particulate form as filler is preferable. The particle diameter in such cases is preferably 0.001-30 μm, more preferably 0.01-25 μm, and still more preferably 0.5-20 μm. If the particle diameter falls below the lower limit of the above-mentioned numerical range, the curing time is extremely short and handling may be difficult. If the particle diameter exceeds the upper limit of the above-mentioned numerical range, adhesive performance may be reduced.

In the present invention, the particle diameter of the TTCP used in the calcium-containing material (D) is normally 0.5-30 μm, more preferably 1-25 μm, and still more preferably 10-20 μm. The particle diameter of the DCP is normally 0.001-30 μm, more preferably 0.05-20 μm, and still more preferably 0.1-15 μm. If the particle diameters fall below the lower limits of the above-mentioned numerical ranges, the curing time is extremely short and handling may be difficult. If the particle diameters exceed the upper limits of the above-mentioned numerical ranges, the curing time is long, and handleability and adhesive performance may be deteriorated.

The existence form of the TTCP and DCP is not particularly restricted. They may form particles in combination where each particle substantially contains the calcium materials in the above-mentioned weight ratio. As long as the total weight ratio falls in the above range, such particles may vary from one another in weight ratio of TTCP and DCP.

The usage amount of the calcium-containing material (D) in the dental composition of the first embodiment of the present invention, that is, the total usage amount of TTCP and DCP, is 0.2-297 parts by weight, and preferably 0.3-248 parts by weight, relative to a total of 100 parts by weight of the components (A) and (B) in the dental composition of the first embodiment of the present invention.

Furthermore, the calcium-containing material (D) is used normally at 1-99 parts by weight, preferably 2-98 parts by weight, and more preferably 5-95 parts by weight, relative to a total of 100 parts by weight of the filler (C) and the calcium-containing material (D). At less than 1 part by weight, which is below the lower limit of the range, the conversion into hydroxyapatite after application is not sufficient and adhesive properties will not be improved. At more than 99 parts by weight, which is above the upper limit of the range, the calcium-containing material will react with the acidic groups in the polymerizable monomer (A) before it is converted into hydroxyapatite, which can also result in reduced adhesive performance.

The component (E) in the dental composition of the first embodiment of the present invention is a polymerization initiator. Examples of the polymerization initiators (E) include organic boron compounds, organic peroxides, inorganic peroxides, redox metal compounds, and photopolymerization initiators.

Examples of the organic boron compounds include trialkylborane compounds such as triethylborane, tri(n-propyl)borane, triisopropylborane, tri(n-butyl)borane, tri(s-butyl)borane, triisobutylborane, tripentylborane, trihexylborane, trioctylborane, tridecylborane, tridodecylborane, tricyclopentylborane, tricyclohexylborane and butyldicyclohexylborane;

alkoxyalkylborane compounds such as butoxydibutylborane;

dialkylboranes such as diisoamylborane and 9-borabicyclo[3.3.1]nonane;

aryl borate compounds such as sodium tetraphenylborate, tetraphenylborate triethanolamine salt, tetraphenylborate dimethyl-p-toluidine salt and tetraphenylborate ethyl dimethylaminobenzoate; and

partially oxidized trialkylborane compounds such as partially oxidized tributylborane.

Examples of the organic peroxides include diacetyl peroxide, dipropyl peroxide, dibutyl peroxide, dicaproyl peroxide, dilauryl peroxide, benzoyl peroxide, p,p′-dichlorobenzoyl peroxide, p,p′-dimethoxybenzoyl peroxide, p,p′-dimethylbenzoyl peroxide and p,p′-dinitrodibenzoyl peroxide.

Examples of the inorganic peroxides include ammonium persulfate, potassium persulfate, potassium chlorate, potassium bromate and potassium superphosphate.

Examples of the redox metal compounds include nitrates, chlorides and acetylaceto salts of transition metals such as copper, iron and cobalt.

Compounds which can initiate polymerization of polymerizable monomers upon irradiation with visible light are preferably used as the photopolymerization initiators. Examples include benzoins such as benzoin, benzoin methyl ether, benzoin ethyl ether and benzoin isopropyl ether;

α-diketones such as benzyl, 4,4′-dichlorobenzyl, diacetyl, α-cyclohexanedione, d,l-camphorquinone, camphorquinone-10-sulfonic acid and camphorquinone-10-carboxylic acid;

diphenyl monoketones such as benzophenone, methyl benzoylbenzoate and hydroxybenzophen one;

thioxanthones such as 2,4-diethyl thioxanthone and 2-isopropyl thioxanthone; and

acyl phosphineoxides such as 2,4,6-trimethylbenzoyldiphenyl phosphineoxide.

Of these, the tributyl borane compounds and partially oxidized tributyl borane compounds are preferably used, and the most preferred organic boron compounds are the partially oxidized tributyl borane compounds.

These compounds may be used singly or as a combination of 2 or more thereof.

The polymerization initiator component (E) in the dental composition of the first embodiment of the present invention may be used normally at 0.5-11 parts by weight, and preferably 1-10 parts by weight, relative to a total of 100 parts by weight of the components (A) and (B) which form the dental composition of the first embodiment of the present invention.

If the photopolymerization initiator is used in the dental composition of the first embodiment of the present invention, the photopolymerization initiator is used normally at 0.1-9.1 wt. %, and preferably 0.2-4.8 wt. %, relative to 100 wt. % of the usage amount of the polymerization initiators (E).

When the photopolymerization initiator is used as polymerization initiator (E) in the dental composition of the first embodiment of the present invention, a reducing compound may be used in combination while still achieving the advantages of the invention. Examples of the reducing compounds include organic reducing compounds such as N,N-dimethylaniline, N,N-dimethyl-p-toluidine, N,N-diethyl-p-toluidine, N,N-diethanol-p-toluidine, N,N-dimethyl-p-t-butylaniline, N,N-dimethylanisidine, N,N-dimethyl-p-chloroaniline, N,N-dimethylaminoethyl (meth)acrylate, N,N-diethylaminoethyl (meth)acrylate, N,N-dimethylaminobenzoic acid and alkyl esters thereof, and salts thereof, N,N-diethylaminobenzoic acid and alkyl esters thereof, and salts thereof, N,N-dimethylaminobenzaldehyde, N-phenylglycine and salts thereof, N-tolylglycine and salts thereof, N,N-(3-(meth)acryloyloxy-2-hydroxypropyl)phenylglycine and salts thereof.

The reducing compound is normally used in an amount 0.5-3.0 times that of the photopolymerization initiator used.

The dental composition of the first embodiment of the present invention may optionally contain a polyfunctional (meth)acrylate (F).

Examples of the polyfunctional (meth)acrylates include:

glycerol di(meth)acrylate;

di(meth)acrylates of butanetriols such as trimethylolpropane;

di(meth)acrylates and tri(meth)acrylates of butanetetraols such as meso-erythritol;

di(meth)acrylates of pentanetriols;

di(meth)acrylates and tri(meth)acrylates of pentanetetraols such as tetramethylolmethane;

polyfunctional (meth)acrylates from xylitol and isomers thereof having 1-2 hydroxyl groups;

di(meth)acrylates of hexanetriols;

di(meth)acrylates and tri(meth)acrylates of hexanetetraols;

polyfunctional (meth)acrylates from hexanepentols having 1-2 hydroxyl groups;

polyfunctional (meth)acrylates from hexanehexyls having 1-2 hydroxyl groups; and

polyfunctional (meth)acrylates represented by formula (3) below:

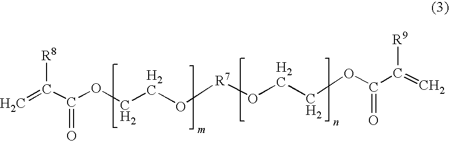

(In formula (3), R⁷ is a divalent aromatic or cycloalkyl residue containing at least 1 aromatic ring and optionally containing an oxygen atom or sulfur atom in the molecule, and is preferably any of the groups selected from a set of chemical formulae (4) below:

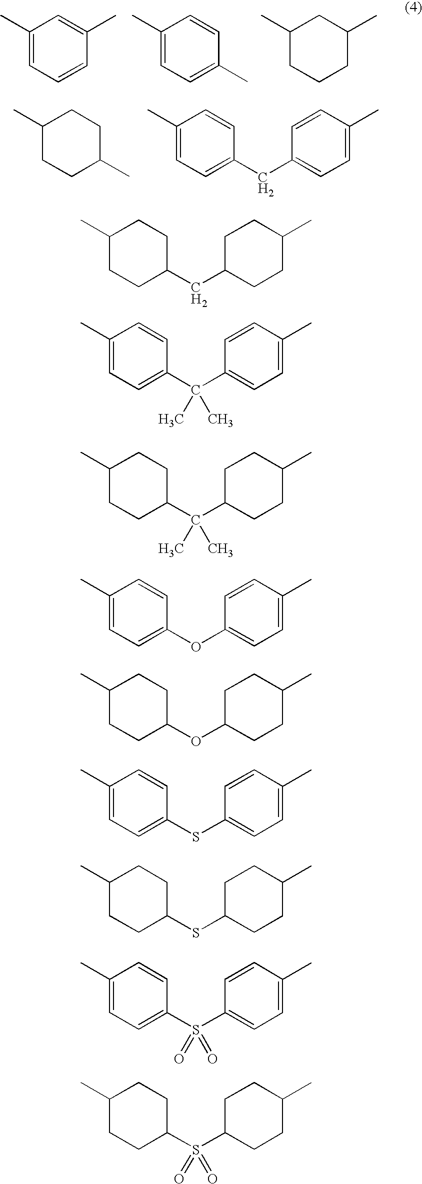

R⁸ and R⁹ represent each independently a hydrogen atom or a methyl group, and n and m are positive integers). Of these polyfunctional (meth)acrylates, those with 2-3 functional groups are preferable, and those having 2 functional groups are more preferable. The presence of many functional groups can cause disadvantages depending on the amount added, such as increased degree of crosslinking in the cured product of the composition, and consequent low softness of the cured product and reduced adhesion to dentin and dental metals.

The polyfunctional (meth)acrylate (F) may be contained in the dental composition of the first embodiment of the present invention at 1-30 parts, preferably 1-20 parts, and most preferably 2-9 parts, based on a total of 100 parts of the components (A), (B) and (F). If the content of the polyfunctional (meth)acrylate (F) is small, the curing will not be accelerated sufficiently. When the content of the polyfunctional (meth)acrylate (F) is large, there is a tendency that the curing reaction proceeds so fast that undue rush is caused to the practitioner. In addition, the water-absorbing properties of the cured product is increased to reduce the adhesive durability to metals or hard tissue such as dentin or bone; and there is a tendency that the cured product of the composition has too high degree of crosslinking and consequent small softness.

In general, a monomer free of acidic groups (F1) is preferably used as the polyfunctional (meth)acrylate (F), but a monomer containing acidic groups (F2) may also be used. In such case, the total content of the components (A) and (F2) is preferably 16-70 parts by weight, more preferably 18-50 parts by weight, and still more preferably 20-40 parts by weight, based on a total of 100 parts by weight of the components (A), (B) and (F2).

The dental composition of the first embodiment of the present invention may optionally contain an inorganic filler or an organic/inorganic composite filler (G).

Examples of the inorganic fillers include metal oxide powders such as zirconium oxide, bismuth oxide, titanium oxide, zinc oxide and aluminum oxide, metal salt powders such as calcium carbonate, bismuth carbonate, zirconium phosphate and barium sulfate, glass fillers such as silica glass, aluminum-containing glass, barium-containing glass, strontium-containing glass and zirconium silicate glass, fillers having sustained silver-release properties, and fillers having sustained fluorine-release properties. These inorganic fillers may be used singly or as a combination thereof.

In order to obtain strong bonding between the inorganic filler and resins, it is preferable to use inorganic fillers that are surface treated by silane treatment or polymer coating.

Furthermore, inorganic/organic composite fillers may be used. The inorganic/organic composite fillers include TMPT fillers (obtained by mixing and polymerizing trimethylol propane methacrylate and a silica filler, and then crushing the polymer).

These inorganic fillers and inorganic/organic composite fillers may be used singly or as a combination thereof.

As mentioned above, in the present invention, the average particle diameter of these particles is preferably 0.001-30 μm, and particularly preferably 0.01-25 μm, in order to reduce the film thickness and improve the repairing effect of the dental composition.

The filler (G) can preferably be used in the dental composition of the first embodiment of the present invention at 15-85 wt. %, more preferably 20-80 wt. %, and still more preferably 25-75 wt. %, based on a total of 100 wt. % of the components (A), (B), (C), (D) and (F) (However, the content of (F) can be 0).

The dental composition of the first embodiment of the present invention may optionally contain a dyestuff and/or pigment (H). Examples of the dyestuffs and/or pigments include phloxine BK, acid red, fast acid magenta, phloxine B, fast green FCF, rhodamine B, basic fuchsin, acidic fuchsin, eosine, erythrosine, safranine, rose bengal, Boehmer's Hematoxylin, gentiana violet, sodium copper chlorophyllin, laccaic acid, sodium fluorescein, cochineal, and shisonin, talc and titanium white. These dyestuffs and/or pigments may be used singly or as a combination of 2 or more thereof.

The dental composition of the first embodiment of the present invention may optionally contain a compound (I) which releases fluoride ions in water. Any compounds can be used as the compound having sustained fluoride ion-release properties (I) as long as the compound releases soluble active fluoride ions in the composition. Examples include disodium fluorophosphate, tin fluoride, sodium fluoride, potassium fluoride, sodium silicofluoride, fluoroaluminosilicate glass and ammonium fluoride, with sodium fluoride, calcium fluoride, sodium monofluorophosphate and stannous fluoride being preferable. The blending quantity thereof is appropriately determined taking into account the usage amount and application frequency of the dental composition of the first embodiment, as well as acid resistance, remineralization efficacy and the effect on the human body. By blending the compound having sustained fluoride ion-release properties (I), improved resistance of hard tissue to acids can be expected. The component (I) is preferably contained at 0.0001-5 wt. %, more preferably 0.001-2 wt. %, and particularly preferably 0.01-1 wt. %, in terms of fluoride ion concentration in the composition. If the concentration is below this range, i.e., less than 0.0001 wt. %, the desired dentin's acid resistance and remineralization effect are not sufficiently achieved. If the concentration exceeds 5 wt. %, there are concerns over harm to the human body.

When any of the compounds used in the dental composition of the first embodiment corresponds to a plurality of the components, the weight of that compound is divided by the number of the components to which it corresponds, and the quotient is used as the weight of each component.

For example, if Compound z corresponds to 2 components (Components X and Y) and its weight is Z g, the contents of Component X and Component Y shall be each half of the weight of Compound Z, namely, Z/2 g and Z/2 g respectively.

By way of another example, if Compound z corresponds to 3 components (Components W, X and Y) and its weight is Z g, the contents of Components W, X and Y shall be each one third of the weight of Compound z, namely, Z/3 g, Z/3 g and Z/3 g respectively.

As described above, the dental composition of the first embodiment of the present invention comprises:

(A) the compound having at least 1 acidic group and polymerizable group in the molecule,

(B) the compound having 1 polymerizable group but not having an acidic group in the molecule,

(C) the filler comprising poly(meth)acrylate particles,

(D) the calcium-containing material containing tetracalcium phosphate (TTCP) and dicalcium phosphate (DCP), and

(E) the polymerization initiator. The dental composition of the first embodiment is constituted by these components in the following amounts: the component (A) in 16-70 parts by weight based on a total of 100 parts by weight of the components (A) and (B), the component (B) in 84-30 parts by weight based on a total of 100 parts by weight of the components (A) and (B), the component (C) in 0.2-297 parts by weight relative to a total of 100 parts by weight of the components (A) and (B), and the component (D) in 0.2-297 parts by weight relative to a total of 100 parts by weight of the components (A) and (B).

Here, the phrases “constituted by” is not limited to the meaning of mixing all the components in the above amounts long before use, and should be understood to mean that the components may be separately stored in a sealed state as appropriate and be mixed together at the time of use. That is, an aspect of the present invention is a dental adhesive composition that is used by blending the components in the above-mentioned weight ratios at the time of use.

The following modes can be illustrated as specific modes of storing the dental adhesive composition of the first embodiment.

(1) The polymerizable monomer which contains acidic groups (A), the polymerizable monomer which does not contain acidic groups (B) and the polymerization initiator (E) are all packaged separately from each other.

(2) The calcium-containing material (D) is packaged separately from each of the polymerizable monomer which contains acidic groups (A), the polymerizable monomer (B) and the polymerization initiator (E).

(3) The polymerizable monomer which contains acidic groups (A) and the polymerizable monomer (B) are packaged separately from the filler (C) comprising poly(meth)acrylate.

(4) The filler (C) comprising poly(meth)acrylate and the calcium-containing material (D) are blended in advance.

The reasons for storing the dental composition of the first embodiment of the present invention as described above are as follows.

If the polymerizable monomer which contains acidic groups (A), the polymerizable monomer (B) and the polymerization initiator (E) are stored together, there are concerns over a polymerization reaction occurring during storage, which would prevent practical use. Thus, it is preferable that these components should be stored in separate packaging. Because the calcium-containing material (D) is often in particulate form with a higher specific gravity than the other components, if the material is together with the polymerizable monomer which contains acidic groups (A), the polymerizable monomer (B) or the polymerization initiator (E) which are often liquids having a lower specific gravity, sedimentation and separation occur during storage, leading to particle aggregation. If the polymerizable monomer which contains acidic groups (A), the polymerizable monomer (B) and the filler (C) comprising poly(meth)acrylate are stored together, the presence of the compounds having polymerizable groups is likely to cause swelling and fusion of poly(meth)acrylates, and these components may substantially lose flowability. Because the filler (C) comprising poly(meth)acrylate and the calcium filler (D) are both preferably in the form of non-liquid dry powder or particles, these components can be used as homogeneous powder by being mixed homogeneously in advance. Such preliminary mixing enables the elimination of careful mixing for actual use.

If tetracalcium phosphate (TTCP) and dicalcium phosphate (DCP) which compose the calcium-containing material (D) are together with water, they are converted into hydroxyapatite. The conversion into hydroxyapatite does not substantially proceed when these are present in a dry state or in a non-aqueous solvent. Thus, these materials can be stored as a mixture under such conditions.

The dental composition of the first embodiment of the present invention may be used for example as follows: the components that are stored as described in (1) to (4) above are taken out on a mixing dish, mixed together at the time of use, and applied to the affected area by means of a brush or the like.

A description of the dental composition of the second embodiment will now be given.

The dental composition of the second embodiment of the present invention is a dental adhesive composition containing:

(A) a compound having at least 1 acidic group and polymerizable group in the molecule,

(J) a polymerizable monomer having at least 1 hydroxyl group in the molecule,

(K) a compound represented by formula (1) and/or formula (2) below:

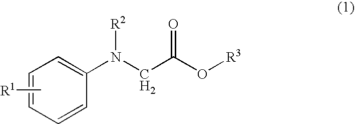

(Wherein R¹ and R² represent each independently a hydrogen atom or an alkyl group optionally containing a functional group or a substituent group, and R³ represents a hydrogen atom or a metal atom.);

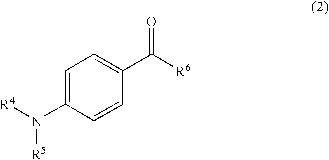

(Wherein R⁴ and R⁵ represent each independently a hydrogen atom or an alkyl group, and R⁶ represents a hydrogen atom, or an alkyl group or alkoxyl group optionally containing a functional group or a substituent group.), and

(D) a calcium filler containing tetracalcium phosphate (TTCP) and dicalcium phosphate (DCP).

The dental composition of the second embodiment contains: (A) the compound having at least 1 acidic group and polymerizable group in the molecule, (J) the polymerizable monomer having at least 1 hydroxyl group in the molecule, (K) the compound represented by formula (1) and/or formula (2) above, and (D) the calcium filler containing tetracalcium phosphate (TTCP) and dicalcium phosphate (DCP). In detail, the use of the calcium filler (D) containing the specific calcium-containing compounds, i.e., tetracalcium phosphate (TTCP) and dicalcium phosphate (DCP), promotes the remineralization of dentin around the affected area.

The component (A) used in the dental composition of the second embodiment of the present invention is the same as the component (A) in the dental composition of the first embodiment described above.

In the dental composition of the second embodiment, 4-(meth)acryloxyethyl trimellitic acid and/or an anhydride thereof is most preferable as the polymerizable monomer containing at least 1 acidic group.

In the dental composition of the second embodiment, the component (A) is contained at 1-50 wt. %, and preferably 1-30 wt. %, based on a total of 100 wt. % of the components (A), (J) and (K). By using this amount of the polymerizable monomer (A) which contains acidic groups in the molecule, the dental composition shows good adhesion to hard tissue, typified by dentin, which is an adherend.

The component (J) in the dental composition of the second embodiment of the present invention is a polymerizable monomer containing at least 1 hydroxyl group in the molecule. The polymerizable monomer which contains the hydroxyl groups may also contain functional groups such as carboxyl groups, phosphate groups, sulfo groups, amino groups and glycidyl groups.

Examples of such monomers which contain (meth)acryloyl groups include monohydroxyalkyl (meth)acrylates such as 2-hydroxyethyl (meth)acrylate, 2- or 3-hydroxypropyl (meth)acrylate, 4-hydroxybutyl (meth)acrylate, 5-hydroxypentyl (meth)acrylate, 6-hydroxyhexyl (meth)acrylate and 10-hydroxydecyl (meth)acrylate;

dihydroxyalkyl (meth)acrylates such as 1,2- or 1,3- or 2,3-dihydroxypropyl (meth)acrylate;

poly(alkylene glycol) (meth)acrylates such as di(ethylene glycol) mono(meth)acrylate, tri(ethylene glycol) mono(meth)acrylate, penta(ethylene glycol) mono(meth)acrylate, poly(ethyleneglycol) mono(meth)acrylate and di(propylene glycol) mono(meth)acrylate;

hydroxyl group-containing (meth)acrylamides such as methylol (meth)acrylamide, (2,3-dihydroxypropyl) (meth)acrylamide and N-(1,3-dihydroxypropyl) (meth)acrylamide; and

adducts of glycidyl (meth)acrylates and aliphatic or aromatic polyols (including phenol), such as 2-hydroxy-3-phenoxypropyl (meth)acrylate, 2-hydroxy-3-naphthoxypropyl (meth)acrylate, and adduct reaction product of 1 mole of bisphenol A and 2 moles of glycidyl (meth)acrylate. These polymerizable monomers may be used singly or as a combination thereof. Of these, 2-hydroxyethyl (meth)acrylate is most preferable.

The component (J) is contained in the dental adhesive composition of the second embodiment at 1-98.99 wt. %, and preferably 3-90 wt. %, based on a total of 100 wt. % of the above-mentioned components (A) and (J) and the below-mentioned component (K).

The component (K) in the dental composition of the second embodiment is represented by formula (1) and/or formula (2) below:

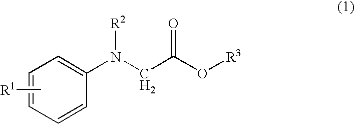

(In formula (1) above, R¹ and R² represent each independently a hydrogen atom or an alkyl group optionally containing a functional group or a substituent group, and R³ represents a hydrogen atom or a metal atom.)

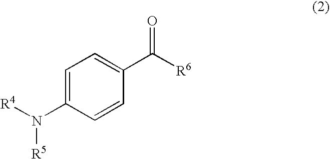

(In formula (2) above, R⁴ and R⁵ represent each independently a hydrogen atom or an alkyl group, and R⁶ represents a hydrogen atom, or an alkyl or alkoxyl group optionally containing a functional group or a substituent group.). This component is used to improve curability and adhesive performance to teeth.

Specific examples of the compounds represented by formula (1) include N-phenylglycine, N-tolylglycine, N-(3-(meth)acryloxy-2-hydroxypropyl)-N-phenylglycine and/or salts of these compounds. Of these, N-phenylglycine and/or salts thereof are preferably used.

Specific examples of the compounds represented by formula (2) include aliphatic alkylaminobenzoic acids and alkyl esters thereof represented by N,N-dimethylaminobenzoic acid and alkyl esters thereof, N,N-diethylaminobenzoic acid and alkyl esters thereof, N,N-dipropylaminobenzoic acid and alkyl esters thereof, N-isopropylaminobenzoic acid and alkyl esters thereof, N-isopropyl-N-methylaminobenzoic acid and alkyl esters thereof; aliphatic alkylaminobenzaldehydes represented by N,N-dimethylaminobenzaldehyde, N,N-diethylaminobenzaldehyde, N,N-dipropylaminobenzaldehyde and N-isopropyl-N-methylaminobenzaldehyde; and aliphatic alkylaminoacetylbenzenes and aliphatic alkylaminoacylbenzenes represented by N,N-dimethylaminoacetylbenzene, N,N-diethylaminoacetylbenzene, N,N-dipropylaminoacetylbenzene, N-isopropylaminoacetylbenzene and N-isopropyl-N-methylaminoacetylbenzene.

These compounds may be used singly or as a combination of 2 or more thereof.

The component (K) is used in the dental composition of the second embodiment at 0.01-30 wt. %, and preferably 0.05-25 wt. %, based on a total of 100 wt. % of the above-mentioned components (A), (J) and (K).

The component (D) used in the dental composition of the second embodiment of the present invention is the same as the component (D) in the dental composition of the first embodiment.

The component (D) is used in the dental composition of the second embodiment normally at 15-95 parts by weight, and preferably 20-90 parts by weight, relative to a total of 100 parts by weight of the above-mentioned components (A), (J) and (K).

If the content is less than 15 parts by weight, which is below the lower limit of the range, the conversion into hydroxyapatite after application is not sufficient and adhesive properties will not be improved. If the content exceeds 95 parts by weight, which is above the upper limit of the range, the component will react with the component (A) before it is converted into hydroxyapatite, which can also result in reduced adhesive performance.

The dental composition of the second embodiment of the present invention may optionally contain a polymerization initiator (E. Compounds similar to the components (E) in the dental composition of the first embodiment may be used as the polymerization initiator (E).

The component (E) is used in the dental composition of the second embodiment normally at 0.01-50 wt. %, preferably 0.05-40 wt. %, and more preferably 0.1-20 wt. %, based on a total of 100 wt. % of the components (A), (J), (K), (E) and optional (F) (However, the content of the below-mentioned component (F) can be 0.).

The dental composition of the second embodiment of the present invention may optionally contain a polyfunctional (meth)acrylate (F).

Compounds similar to the components (F) in the dental composition of the first embodiment may be used as the polyfunctional (meth)acrylate (F).

The component (F) is used in the dental composition of the second embodiment normally at 5-70 wt. %, more preferably 10-65 wt. %, and still more preferably 15-60 wt. %, based on a total of 100 wt. % of the components (A), (J), (K), (E) and (F) (However, the content of the component (E) can be 0.).

If the amount of the polyfunctional (meth)acrylate (F) is small, the curing will not be accelerated sufficiently. If the amount of the component (F) is large, there is a tendency that the curing reaction proceeds so fast that undue rush is caused to the practitioner. In addition, the water-absorbing properties of the cured product is increased to reduce the adhesive durability to metals or hard tissue such as dentin or bone; and there is a tendency that the cured product of the composition has too high degree of crosslinking and consequent small softness.

The dental composition of the second embodiment of the present invention may optionally contain at least one filler (L) selected from the group consisting of organic fillers, inorganic fillers and organic/inorganic composite fillers (L).

The organic fillers include poly(alkyl (meth)acrylate)s, polyolefins, poly(vinyl acetate)s, polyethylene glycol)s and poly(propyleneglycol)s.

Examples of the monomers for the poly(alkyl (meth)acrylates) include (meth)acrylate monomers such as methyl (meth)acrylate, ethyl (meth)acrylate, propyl (meth)acrylate, butyl (meth)acrylate, cyclohexyl (meth)acrylate and benzyl (meth)acrylate. The poly(alkyl (meth)acrylates) may be copolymerized with small quantities of crosslinkable monomers as required. Examples of the crosslinkable monomers include polyfunctional monomers such as ethylene glycol di(meth)acrylate, triethylene glycol di(meth)acrylate and butadiene.

Examples of the monomers for the polyolefins include ethylene and propylene. The polyolefins may be copolymerized with components such as ethylidenenorbornene, 1,4-hexadiene and dicyclopentadiene as required.

The inorganic fillers include metal oxide powders such as zirconium oxide, bismuth oxide, titanium oxide, zinc oxide and aluminum oxide; metal salt powders such as calcium carbonate, bismuth carbonate, zirconium phosphate and barium sulfate; glass fillers such as silica glass, aluminum-containing glass, barium-containing glass, strontium-containing glass and zirconium silicate glass; fillers having sustained silver-release properties and fillers having sustained fluorine-release properties.

In order to obtain strong bonding between the inorganic filler and resins, it is preferable to use inorganic fillers that are surface treated by silane treatment or polymer coating.

Furthermore, inorganic/organic composite fillers may be used as the fillers in the invention. The inorganic/organic composite fillers include TMPT fillers (obtained by mixing and polymerizing trimethylol propane methacrylate and a silica filler, and then crushing the polymer).

These organic fillers, inorganic fillers and inorganic/organic composite fillers may be used singly or as a combination thereof.

The fillers (L) used in the dental composition of the second embodiment are preferably composed of 40-80 wt. % of a zirconium oxide filler having an average particle diameter of 0.05-10 μm, 10-30 wt. % of a spherical silica filler having an average particle diameter of 1-10 μm, and 10-30 wt. % of an organic composite filler having an average particle diameter of 1-30 μm. Furthermore, the zirconium oxide used here is preferably coated with a polymer that is soluble in the component (J), for example, polymethyl methacrylate (PMMA) or polyvinyl acetate (PVAc), and has an average particle diameter of 5-30 μm, particularly 1-30 μm.

The usage amount of the component (L) in the dental composition of the second embodiment is normally 15-85 wt. %, more preferably 20-80 wt. %, and still more preferably 25-75 wt. %, based on a total of 100 wt. % of the components (A), (J), (K), (D), (E), (F) and (L) (However, the content of at least either of the components (E) and (F) can be 0.).

The dental composition of the second embodiment of the present invention may optionally contain a dyestuff and/or pigment (H). Compounds similar to the components (H) in the dental composition of the first embodiment may be used as the dyestuff and/or pigment (H).

The dental composition of the second embodiment of the present invention may optionally contain a compound (I) which releases fluoride ions in water. Compounds similar to the components (I) in the dental composition of the first embodiment may be used as the component (I).

The amount thereof is appropriately determined taking into account the usage amount and application frequency of the dental composition of the second embodiment, as well as acid resistance, remineralization efficacy and effect on the human body. By blending the component (I), improved resistance of hard tissue to acids can be expected. The component (I) is preferably used at 0.0001-5 wt. %, more preferably 0.001-2 wt. %, and particularly preferably 0.01-1 wt. %, in terms of fluoride ion concentration in the composition. If the concentration is below this range, i.e., less than 0.0001 wt. %, the desired dentin's acid resistance and remineralization effect are not sufficiently achieved. If the concentration exceeds 5 wt. %, there are concerns over harm to the human body.

The dental composition of the second embodiment of the present invention may optionally contain a polymerization inhibitor (N) to ensure storage stability. Heretofore-known polymerization inhibitors may be used as the polymerization inhibitor (N), with examples including 4-methylphenol and 2,6-di-t-butylcresol.

When any of the compounds used in the dental composition of the second embodiment corresponds to a plurality of the components, the weight of that compound is divided by the number of the components to which it corresponds, and the quotient is used as the weight of each component. For example, if Compound z corresponds to 2 components (Components X and Y) and its weight is Z g, the contents of Component X and Component Y shall be each half of the weight of Compound z, namely, Z/2 g and Z/2 g respectively. By way of another example, if Compound z corresponds to 3 components (Components W, X and Y) and its weight is Z g, the contents of Components W, X and Y shall be each one third of the weight of Compound z, namely, Z/3 g, Z/3 g and Z/3 g respectively.

The dental composition of the second embodiment of the present invention may optionally contain a solvent. Solvents similar to those described in the dental composition of the first embodiment may be used as the solvent.

In an exemplary mode of using the dental composition of the second embodiment, the above-mentioned components (A) to (J) are blended in advance and then applied to dental tissue. If there are fears that the mixture of these components will change its form or performance over a long period of time to fail to achieve the advantages of the present invention, the components may be separately stored individually or in appropriate combination and be mixed together at the time of use to constitute the dental composition.

Here, the word “constitute” is not limited to the meaning of mixing all the components in the above amounts long before use, and should be understood to mean that the components may be separately stored in a sealed state as appropriate and be mixed together at the time of use. That is, an aspect of the present invention is a dental composition that is used by blending the components in the above-mentioned weight ratios at the time of use.

The following are exemplary modes of storing the dental composition of the second embodiment.

(1) There are mixed at least 2 components selected from the group consisting of the acidic group-containing polymerizable monomer (A), the hydroxyl group-containing polymerizable monomer (J) and the polyfunctional (meth)acrylate compound (F).

(2) There are mixed at least 2 components selected from the group consisting of the compound (K) represented by formula (1) or (2), the calcium filler (D), the polymerization initiator (E), the filler (L) and the fluorine-releasing compound (I).

(3) The acidic group-containing polymerizable monomer (A), the hydroxyl group-containing polymerizable monomer (J) and/or the polyfunctional (meth)acrylate compound (F) is packaged separately from the polymerization initiator (E), the compound (K) represented by formula (1) or (2), the calcium filler (D), the filler (L) and/or the fluorine-releasing compound (I).

(4) The polymerization initiator (E) and/or the compound (K) represented by formula (1) or (2) is packaged separately from the calcium filler (D), the filler (L) and/or the fluorine-releasing compound (I).

The reasons for storing the dental composition as described above are as follows.

The acidic group-containing polymerizable monomer (A), the hydroxyl group-containing polymerizable monomer (J) and the polyfunctional (meth)acrylate compound (F) are often either liquid at ambient temperature or are highly compatible with each other. Therefore, these components will not separate even if blended together in advance, and such preliminary mixing is generally advantageous in that the blending at the time of use can be eliminated. The compound (K) represented by formula (1) or (2), the polymerization initiator (E), the calcium filler (D), the filler (L) and the fluorine-releasing compound (I) are often easily prepared in the form of powder or particles, and are therefore suitably mixed together in the form of powder.

The mixture of the polymerizable monomers (i.e., the acidic group-containing polymerizable monomer (A), the hydroxyl group-containing polymerizable monomer (J) and the polyfunctional (meth)acrylate compound (F)) should be suitably separated from the compound (K) represented by formula (1) or (2), the calcium filler (D), the polymerization initiator (E), the filler (L) and the fluorine-releasing compound (I). Separated from the polymerization initiator (E), the polymerizable monomers are prevented from polymerization during storage. In addition, this packaging mode has advantages that because the calcium filler (D), which is often basic, is separated, the acidic groups in the monomers will not be neutralized or ion exchanged and the intact performance of the composition can be ensured, and that the calcium filler (D), the filler (L) and the fluorine-releasing compound (I), which are poorly soluble, will not cause problems by becoming unhomogeneous or aggregated in the monomer liquid.

In the presence of water, the mixture of tetracalcium phosphate (TTCP) and dicalcium phosphate (DCP) is converted into hydroxyapatite. The conversion into hydroxyapatite does not substantially proceed when these phosphates are present in a dry state, free of water of crystallization, or in a non-aqueous solvent. Thus, these materials as a mixture can be stored under such conditions, and the obtain able dental composition of the second embodiment may be suitably used.

### EXAMPLES

Hereafter, the present invention is described in more detail by examples, but the present invention is not in any way limited by these examples.

### (Explanation of Abbreviations)

The meanings of the abbreviations used in the examples below are as follows.

TTCP: Tetracalcium phosphate (Ca₄(PO₄)₂O)

DCP: Dicalcium phosphate (CaHPO₄)

MMA: Methyl methacrylate

4-META: 4-methacryloxyethyl trimellitic anhydride

PMMA: Poly(methyl methacrylate)

HEMA: 2-hydroxyethyl methacrylate

4-MET: 4-methacryloxyethyl trimellitic acid

P-2M: Bis(2-methacryloxyethyl)phosphate

3G: Tri(ethylene glycol)dimethacrylate

2.6-E: 2,2-bis(4-methacryloxy poly(ethoxy)phenyl)propane

A-9300: N,N,N-tris(2-acryloxyethyl)isocyanurate

Bis-GMA: 2,2-bis[4-(2-hydroxy-3-methacryloxypropoxy)phenyl]propane

CQ: d,l-camphorquinone

NPGNa: N-phenylglycine sodium salt

EDEAB: Ethyl 4-(N,N-diethylamino)benzoate

(Preparation of Calcium-Containing Material)

TTCP and DCP were each crushed using a planetary ball mill and sieved with a #280 sieve. 36.7 g (0.1 mole) of the sieved TTCP and 13.6 g (0.1 mole) of the sieved DCP were well mixed using the planetary ball mill to give a calcium-containing material. When the average particle diameter of the calcium filler was measured with a laser diffraction scattering particle size distribution analyzer (LA-910, manufactured by Horiba, Ltd.), the average particle diameter was 1 μm and the maximum particle diameter was 10 μm.

In Examples 1-4 and Comparative Examples 1 and 2 below, the adhesive strength to dentin was measured and marginal sealing properties were evaluated as follows.

### (Measurement of Adhesive Strength to Dentin)

A bovine mandibular anterior tooth was polished under running water with #180 emery paper to give a bonding flat dentin surface, and the tooth was washed with water and dried. The surface was masked with an adhesive tape to define a circular bonding area 4.8 mm in diameter. Approximately 0.09 g of a monomer liquid having a predetermined composition was placed in a mixing dish, and about 0.007 g (7.8 parts relative to 100 parts of the monomer liquid) of Super-Bond Catalyst (partially oxidized tributylborane (corresponding to component (E)), manufactured by Sun Medical Co., Ltd.) was added thereto dropwise. They were mixed well with a brush. The brush holding a sufficient amount of the mixture was brought into contact with the powder shown below to attach the powder thereto. The liquid and the powder were mixed by the stirring movement of the adhesive brush. The mixture was applied on the bonding tooth surface, and then a PMMA bonding rod was pressure planted on the bonding surface. After 1 hour, the bonded sample was immersed in distilled water at 37° C. for 24 hours. The tensile adhesive strength was then evaluated at a cross head speed of 2 mm/min, whereby the basic properties of the adhesive material were evaluated.

(Evaluation of Marginal Sealing Properties)

A bovine mandibular anterior tooth was polished under running water with #180 emery paper to give a flat dentin surface, and a dental air turbine was used under running water to form a dental cavity having a diameter of approximately 3 mm and a depth of 2 mm. The surface of the dental cavity was treated for 10 seconds with the dentin surface treating material “Green” (a dental surface treating agent for dentin which had citric acid and ferric chloride as its principal components, manufactured by Sun Medical Co., Ltd.). The tooth was then washed with water and dried. Approximately 0.09 g of a monomer liquid having a predetermined composition (corresponding to component (A) and component (B)) was placed in a mixing dish, and about 0.007 g (7.8 parts by weight relative to 100 parts by weight of the monomer liquid) of Super-Bond Catalyst (partially oxidized tributylborane (corresponding to component (E)), manufactured by Sun Medical Co., Ltd.) was added thereto dropwise. They were mixed with 0.08 g of the below-mentioned powder with use of an adhesive brush. The dental cavity was filled with the mixture, and the filled surface was covered with cellophane (registered trademark). The tooth was left overnight at 37° C. and saturated humidity. Thereafter, the surface of the filler was polished under running water using #600 emery paper to give a flat surface. The packed tooth was then immersed in a 0.3% aqueous basic fuchsin solution for 3 minutes. The tooth removed from the aqueous fuchsin solution was washed with distilled water, and the packed surface was polished again under running water with #100 emery paper. The tooth was observed with a microscope to determine whether fuchsin had permeated from the polished surface toward the dentin. Furthermore, the packed tooth was cut in a direction parallel to the direction in which the cavity extended. The exposed surface was observed with a microscope to determine whether fuchsin had permeated from the surface of the dental cavity to the inner part of the dentin. The teeth that showed no fuchsin permeation in both observations were evaluated as ◯, and those in which fuchsin had permeated were evaluated as X.

### Example 1

In the above-mentioned dentin adhesive strength test and marginal sealing properties test, the monomer liquid used was a mixture of 80 g of MMA and 20 g of 4-META, and the filler used was a mixture of 3 g of the above-mentioned calcium-containing material and 97 g of PMMA (Super-Bond Powder Clear, manufactured by Sun Medical Co., Ltd.) which had been mixed using a planetary ball mill.

The results are shown in Table 1.

### Example 2

In the above-mentioned dentin adhesive strength test and marginal sealing properties test, the monomer liquid used was a mixture of 80 g of MA and 20 g of 4-META, and the filler used was a mixture of 30 g of the above-mentioned calcium filler and 70 g of PMMA (Super-Bond Powder Clear, manufactured by Sun Medical Co., Ltd.) which had been mixed using a planetary ball mill.

The results are shown in Table 1.

### Example 3

In the above-mentioned dentin adhesive strength test and marginal sealing properties test, the monomer liquid used was a mixture of 70 g of MMA and 30 g of 4-META, and the filler used was a mixture of 3 g of the above-mentioned calcium filler and 97 g of PMMA (Super-Bond Powder Clear, manufactured by Sun Medical Co., Ltd.) which had been mixed using a planetary ball mill.

The results are shown in Table 1.

### Example 4

In the above-mentioned dentin adhesive strength test and marginal sealing properties test, the monomer liquid used was a mixture of 70 g of MMA and 30 g of 4-META, and the filler used was a mixture of 30 g of the above-mentioned calcium filler and 70 g of PMMA (Super-Bond Powder Clear, manufactured by Sun Medical Co., Ltd.) which had been mixed using a planetary ball mill.

The results are shown in Table 1.

### Comparative Example 1

In the above-mentioned dentin adhesive strength test and marginal sealing properties test, the monomer liquid used was MMA, and the filler used was a mixture of 3 g of the above-mentioned calcium filler and 97 g of PMMA (Super-Bond Powder Clear, manufactured by Sun Medical Co., Ltd.) which had been mixed using a planetary ball mill. The results are shown in Table 1. The adhesion failure was thought to be due to the non-use of any acidic monomers.

### Comparative Example 2

In the above-mentioned dentin adhesive strength test and marginal sealing properties test, the monomer liquid used was a mixture of 95 g of MMA and 5 g of 4-META, and the filler used was a mixture of 3 g of the above-mentioned calcium filler and 97 g of PMMA (Super-Bond Powder Clear, manufactured by Sun Medical Co., Ltd.) which had been mixed using a planetary ball mill.

The results are shown in Table 1. The low adhesive strength was thought to be due to the low concentration of the acidic monomer, i.e., the consumption of the acidic monomer by reaction with CPC before the monomer diffused into the dentin.

TABLE 1

| | | | Filler component | | | |
|---|---|---|---|---|---|---|
| | | | (%) | | Ad- | |
| | Monomer | | | Calcium- | hesive | Marginal |
| | component (%) | | | containing | strength | sealing |
| Ex. | MMA | 4-META | PMMA | material | (MPa) | properties |
| Ex. 1 | 80 | 20 | 97 | 3 | 6.3 | ◯ |
| Ex. 2 | 80 | 20 | 70 | 30 | 6.0 | ◯ |
| Ex. 3 | 70 | 30 | 97 | 3 | 7.5 | ◯ |
| Ex. 4 | 70 | 30 | 70 | 30 | 7.0 | ◯ |
| Comp. | 100 | 0 | 97 | 3 | 0 | X |
| Ex. 1 | | | | | | |
| Comp. | 95 | 5 | 97 | 3 | 2.0 | ◯ |
| Ex. 2 | | | | | | |

In Examples 5-8 below, the adhesive strength to dentin was measured and marginal sealing properties were evaluated as follows.

### (Measurement of Adhesive Strength to Dentin)

A bovine mandibular anterior tooth was polished under running water with #180 emery paper to give a bonding flat dentin surfacer and the tooth was washed with water and dried. A mold having a diameter of 4.8 mm and a thickness of 1 mm was fixed to the dentin surface with a double-sided tape to define the bonding area. Approximately 0.09 g of a liquid material having a predetermined composition and 0.9 g of a powder material obtained with a planetary ball mill and having a predetermined composition were kneaded on paper. The mixture was then filled in the mold, and the filled surface was covered with a clear polyester film and irradiated by use of a dental visible light irradiator (tradename: Translux CL2 (Heraeus-Kulzer)) for 30 seconds. Next, the polyester film was removed, and the cured product and a PMMA rod having a diameter of 6 mm and a height of 20 mm were bonded together with a dental adhesive (tradename: Super-Bond C&B, manufactured by Sun Medical Co., Ltd.). The unit was left to stand at ambient temperature for 30 minutes, and then immersed in water at 37° C. for 24 hours. The tensile adhesive strength was evaluated at a cross head speed of 1 mm/min, whereby the basic properties of the adhesive material were evaluated.

### (Evaluation of Marginal Sealing Properties)

A bovine mandibular anterior tooth was polished under running water with #180 emery paper to give a flat dentin surface, and a dental air turbine was used under running water to form a dental cavity having a diameter of approximately 3 mm and a depth of 2 mm. The surface of the dental cavity was treated for 10 seconds with “Green Activator” (a dental surface treating agent for dentin which had citric acid and ferric chloride as its principal components (manufactured by Sun Medical Co., Ltd.)). The tooth was then washed with water and dried. 0.09 g of a monomer liquid having a predetermined composition and 0.9 g of a powder material having a predetermined composition were kneaded on paper. The mixture was packed inside the dental cavity, and the packed surface was covered with cellophane (registered trademark). The tooth was left overnight at 37° C. and saturated humidity. Thereafter, the surface of the filler was polished under running water using 4600 emery paper to give a flat surface. The packed tooth was immersed in a 0.3% aqueous basic fuchsin solution for 3 minutes. The tooth removed from the aqueous fuchsin solution was washed with distilled water, and the packed surface was polished again under running water with #100 emery paper. The tooth was observed with a microscope to determine whether fuchsin had permeated from the polished surface toward the dentin. Furthermore, the packed tooth was cut in a direction parallel to the direction in which the cavity extended. The exposed surface was observed with a microscope to determine whether fuchsin had permeated from the surface of the dental cavity to the inner part of the dentin. The teeth that showed no fuchsin permeation in both observations were evaluated as ◯, and those in which fuchsin had permeated were evaluated as X.

### Example 5

A liquid material was obtained by mixing 20 g of 4-MET, 39.5 g of HEMA, 20 g of Bis-GMA, 15 g of 2.6-E, 3 g of 3G and 0.5 g of CQ.

Meanwhile, a powder material comprising 97 g of the above-mentioned calcium filler and 3 g of NPGNa was obtained.

The above-mentioned dentin adhesive strength test and marginal sealing properties test were performed using these materials, resulting in 9 MPa and “◯” respectively.

### Example 6

A liquid material was obtained by mixing 10 g of 4-MET, 2 g of P-2M, 40 g of HEMA, 20 g of A-9300, 10 g of 3G and 0.5 g of CQ.

Meanwhile, a powder material was obtained which comprised 50 g of the above-mentioned calcium filler, 30 g of zirconium oxide having an average particle diameter of 1 μm, 19 g of a spherical silica filler having an average particle diameter of 3 μm and 1 g of NPGNa.

The above-mentioned dentin adhesive strength test and marginal sealing properties test were performed using these materials, resulting in 12 MPa and “O” respectively.

### Example 7

A liquid material was obtained by mixing 20 g of 4-MET, 39.5 g of HEMA, 20 g of Bis-GMA, 15 g of 2.6-E, 5 g of 3G and 0.5 g of CQ.

Meanwhile, a powder material comprising 97 g of the calcium filler and 3 g of EDEAB was obtained.

The above-mentioned dentin adhesive strength test and marginal sealing properties test were performed using these materials, resulting in 8 MPa and “◯” respectively.

### Example 8

A liquid material was obtained by mixing 10 g of 4-MET, 2 g of P-2M, 40 g of HEMA, 20 g of A-9300, 10 g of 3G and 0.5 g of CQ.

Meanwhile, a powder material was obtained which comprised 50 g of the above-mentioned calcium filler, 30 g of zirconium oxide having an average particle diameter of 1 μm, 19 g of a spherical silica filler having an average particle diameter of 3 μm and 1 g of EDEAB.

The above-mentioned dentin adhesive strength test and marginal sealing properties test were performed using these materials, resulting in 9 MPa and “◯” respectively.

1. A dental composition comprising:
(A) a compound having at least 1 acidic group and polymerizable group in the molecule,
(B) a compound having 1 polymerizable group but not having an acidic group in the molecule,
(C) a filler comprising poly(meth)acrylate particles,
(D) a calcium-containing material including tetracalcium phosphate (TTCP) and dicalcium phosphate (DCP), and
(E) a polymerization initiator,
the component (A) being contained at 16-70 parts by weight based on a total of 100 parts by weight of the components (A) and (B), the component (B) being contained at 84-30 parts by weight based on a total of 100 parts by weight of the components (A) and (B), the component (C) being contained at 0.2-297 parts by weight relative to a total of 100 parts by weight of the components (A) and (B), the component (D) being contained at 0.2-297 parts by weight relative to a total of 100 parts by weight of the components (A) and (B).

2. The dental composition as described in claim 1, wherein the molar ratio of tetracalcium phosphate (TTCP) to dicalcium phosphate (DCP) (TTCP/DCP) is within the range 0.33-3.5.

3. The dental composition as described in claim 1, wherein the dental composition further comprises (F) a polyfunctional (meth)acrylate compound.

4. The dental composition as described in claim 1, wherein the dental composition further comprises (G) an inorganic filler or inorganic/organic composite filler.

5. The dental composition as described in claim 1, wherein the dental composition further comprises (H) a dyestuff and/or pigment.

6. The dental composition as described in claim 1, wherein the dental composition further comprises (I) a compound which releases fluoride ions in water.

7. The dental composition as described in claim 1, wherein the compound (A) having at least 1 acidic group and polymerizable group in the molecule is 4-(meth)acryloxyethyl trimellitic acid and/or an anhydride thereof.

8. The dental composition as described in claim 1, wherein the polymerization initiator (E) is a trialkylborane compound.

9. The dental composition as described in claim 1, wherein the polymerization initiator (E) is tributylborane and/or a partial oxide thereof.

10. A dental composition comprising:
(A) a compound having at least 1 acidic group and polymerizable group in the molecule,
(J) a polymerizable monomer having at least 1 hydroxyl group in the molecule,
(K) a compound represented by formula (1) and/or formula (2) below: 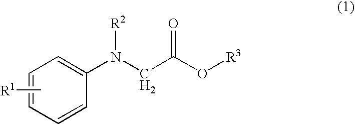
(wherein R¹ and R² represent each independently a hydrogen atom, or an alkyl group optionally containing a functional group or a substituent group, and R³ represents a hydrogen atom or a metal atom); 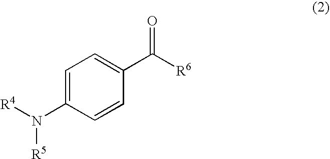
(wherein R⁴ and R⁵ represent each independently a hydrogen atom or an alkyl group, and R⁶ represents a hydrogen atom, or an alkyl group or alkoxyl group optionally containing a functional group or a substituent group), and
(D) a calcium filler including tetracalcium phosphate (TTCP) and dicalcium phosphate (DCP),
the usage amount of the acidic group-containing polymerizable monomer (A) being 1-50 wt. %, the usage amount of the hydroxyl group-containing polymerizable monomer (J) being 1-98.99 wt. %, the usage amount of the compound (K) represented by formula (1) and/or formula (2) being 0.01-30 wt. %, each relative to 100 wt. % of the total usage amount of the acidic group-containing polymerizable monomer (A), the hydroxyl group-containing polymerizable monomer (J) and the compound (K) represented by formula (1) and/or formula (2),
the usage amount of the calcium filler (D) including tetracalcium phosphate (TTCP) and dicalcium phosphate (DCP) being 15-95 parts by weight relative to 100 parts by weight of the total usage amount of the acidic group-containing polymerizable monomer (A), the hydroxyl group-containing polymerizable monomer (J) and the compound (K) represented by formula (1) and/or formula (2).

11. The dental adhesive composition as described in claim 10, wherein the dental composition further comprises a polymerization initiator (E) in addition to the acidic-group containing polymerizable monomer (A), hydroxyl group-containing polymerizable monomer (J), compound (K) represented by formula (1) and/or formula (2), and calcium filler (D), wherein the usage amount of the polymerization initiator (E) which constitutes the dental composition is 0.01-50 wt. % based on a total of 100 wt. % of the acidic-group containing polymerizable monomer (A), the hydroxyl group-containing polymerizable monomer (J), the compound (K) represented by formula (1) and/or formula (2), the polymerization initiator (E) and the polyfunctional (meth)acrylate compound (F) which is optional.

12. The dental composition as described in claim 10, wherein the component (D) has a molar ratio of tetracalcium phosphate (TTCP) to dicalcium phosphate (DCP) (TTCP/DCP) within the range 0.33-3.5.

13. The dental composition as described in claim 10, wherein the dental composition further comprises (F) a polyfunctional (meth)acrylate compound.

14. The dental composition as described in claim 10, wherein the dental composition further comprises (L) at least 1 filler selected from the group consisting of organic fillers, inorganic fillers and organic/inorganic composite fillers.

15. The dental composition as described in claim 10, wherein the dental composition further comprises (H) a pigment.

16. The dental composition as described in claim 10, wherein the dental composition further comprises (I) a compound which releases fluoride ions in water.

17. The dental composition as described in claim 10, wherein the polymerizable monomer (J) having at least 1 hydroxyl group in the molecule is 2-hydroxyethyl (meth)acrylate.

18. The dental composition as described in claim 11, wherein the polymerization initiator (E) is at least 1 polymerization initiator selected from the group consisting of organic peroxides, inorganic peroxides, alkylboranes, partial oxides of alkylboranes, α-diketone compounds, acylphosphineoxides, organic sulfinic acids, organic sulfinates, inorganic sulfur compounds and barbituric acids.

19. The dental composition as described in claim 1, wherein the dental composition is a dental adhesive composition.

20. The dental composition as described in claim 1, which is in a dry state or is in a non-aqueous solvent.

21. The dental composition as described in claim 10, which is in a dry state, is free of water of crystallization, or is in a non-aqueous solvent.

22. The dental composition as described in claim 1, wherein the particle diameter of dicalcium phosphate (DCP) is 0.1 to 15 μm.

23. The dental composition as described claim 10, wherein the particle diameter of dicalcium phosphate (DCP) is 0.1 to 15 μm.
